(19) United States
(12) Patent Application Publication
(10) Pub. No.: US 2026/0154884 A1
(43) Pub. Date: Jun. 4, 2026

(54) CROSS-TERM SPATIOTEMPORAL ENCODING (xSPEN) TECHNIQUES FOR SINGLE-SIDED MRI

(71) Applicant: Promaxo, Inc., Oakland, CA (US)

(72) Inventors: Muller Francis DE MATOS GOMES, Hayward, CA (US); Aleksandar NACEV, San Francisco, CA (US); William Allyn GRISSOM, Nashville, TN (US)

(21) Appl. No.: 19/261,467

(22) Filed: Jul. 7, 2025

Related U.S. Application Data
(63) Continuation of application No. PCT/US2024/010647, filed on Jan. 8, 2024.
(60) Provisional application No. 63/479,162, filed on Jan. 9, 2023.

(51) Int. Cl. G06T 12/20; G06T 15/00; G16H 30/20

(52) U.S. Cl. CPC G06T 12/20; G06T 15/00; G16H 30/20

(57) ABSTRACT

The disclosure provides a single-sided magnetic resonance imaging system containing multiple components. The disclosure also provides methods for generating multi-dimensional images by using the imaging system. The disclosure also provides processes of applying a plurality of xSPEN readout gradient on the single-sided magnetic resonance imaging system disclosed herein.

### CROSS REFERENCE TO RELATED APPLICATIONS

This application is a continuation of International Application No. PCT/US2024/010647, filed Jan. 8, 2024, which claims the benefit of U.S. Provisional Application No. 63/479,162, filed Jan. 9, 2023, each of which are hereby incorporated by reference in their entirety.

### BACKGROUND

The present disclosure is related to medical systems, devices, and methods, particularly those for biomedical imaging, including single-sided or open magnetic resonance (MRI) scanners.

Single-sided or open magnetic resonance imaging (MRI) scanners generally have a permanent or inherent gradient magnetic field along a longitudinal axis extending from the single-sided MRI apparatus into a field of view. The permanent gradient magnetic field can be produced by rare earth magnets and two sets of gradient coils on the face of the permanent magnets. This orientation allows imaging within a field of view above the face of the magnet. By designing a system with this form factor, it is possible to image without having to enclose the region being imaged. So, one may then image without having a patient enter a bore, allowing for the scanner to be used with other medical devices, such as a biopsy robot, for example. It is also more comfortable for claustrophobic patients to be imaged outside an imaging bore of a conventional, enclosed MRI scanner. Single-sided MRIs can also be portable and can image anything positioned within the field of view.

The use of a surface gradient coil with a single-sided scanner, though generally needed for one sided scanning, can result in a changing field of view along the Z axis, a drifting echo, and/or ultimately in the truncation of k-space, which may cause blurring and effectively limit the image quality obtained by the single-sided MRI scanner. Therefore, there are needs for improved sample acquisition for single-sided or open MRI scanners.

### SUMMARY

There are several techniques for collecting dynamic magnetic resonance (MR) images. Fast pulse sequences can be used to rapidly sample the entirety of k space thereby generating an image acquired over a small time window. For example, a spiral trajectory gradient echo sequence can be used to sample the entirety of k space in tens of milliseconds. Another approach is to use a small flip angle excitations to rapidly collect lines in k space. These approaches can also be combined with under sampling to collect images even more rapidly. However, each of these techniques assumes a strong homogeneous magnetic field and tends to breakdown as the homogeneity of the main magnetic field is reduced.

Some methods for rapidly collecting images in an inhomogeneous field also exist, with the most relevant one being xSPEN. Different kinds of xSPEN pulse sequences are described in Zhang, Zhiyong, et al. “Single-Scan MRI with Exceptional Resilience to Field Heterogeneities” Magnetic Resonance in Medicine, vol. 77, 2017, pp. 623634 (hereinafter, “Zhang, Zhiyong, et al.”), which is herein incorporated by reference in its entirety. In some cases, a pulse sequence resembling a portion of the xSPEN sequence disclosed in the supplemental information of the Zhang, Zhiyong, et al., in section 5.4, can be used. The supplemental informal of the Zhang, Zhiyong, et al. is also herein incorporated by reference in its entirety. These sequences allow for one to define an imaging axis without interference from a permanent gradient.

In low SNR environments, it may be necessary to signal average or sample k space with a short echo time to improve SNR. Making these adjustments to existing pulse sequences can slow down image acquisition.

Existing methodologies are also not designed for inhomogeneous magnetic fields. Collecting all of k space with a single spiral is not feasible in a system with a permanent gradient. The permanent gradient may limit the trajectories that can be achieved with the scanner.

To address these issues, xSPEN can be used to rapidly image in a permanent gradient, but existing versions are also not optimal for single sided low field systems. At low magnetic fields with strong permanent gradients, SNR is much lower, which limits the applicability of xSPEN.

In one aspect, disclosed herein is a single-sided magnetic resonance imaging system comprising: a housing comprising: a front surface; a permanent magnet for providing a static magnetic field that extends from the permanent magnet relative to a first axis into a region of interest, wherein the first axis is perpendicular to the permanent magnet; a radio frequency transmit coil; and a single-sided gradient coil set, wherein the radio frequency transmit coil and the single-sided gradient coil set are positioned proximate to the front surface; a radio frequency receive coil; a power source, wherein the power source is configured to flow current through at least one of the radio frequency transmit coil or the single-sided gradient coil set to generate an electromagnetic field in the region of interest, wherein at least a portion of the region of interest resides outside the front surface; and a control circuit configured to: transmit an excitation pulse with the radio frequency transmit coil; transmit a first refocusing pulse with the radio frequency transmit coil; apply a first xSPEN readout gradient, during the first refocusing pulse, along a second axis orthogonal to the first axis with the single-sided gradient coil set; perform phase encoding along a third axis orthogonal to the first axis and orthogonal to the second axis with the single-sided gradient coil set; transmit a second refocusing pulse with the radio frequency transmit coil; apply a second xSPEN readout gradient, during the second refocusing pulse, along the second axis with the single-sided gradient coil set; and receive data with the radio frequency receive coil. In some embodiments, the control circuit is further configured to: transmit any number of refocusing pulses with the radio frequency transmit coil before, between, or after the refocusing pulses that occur simultaneously with the xSPEN readout gradients. In some embodiments, the control circuit is further configured to: generate an encoding matrix that is used to generate an image, wherein the encoding matrix accounts for a residual linear phase and a residual quadratic phase. In some embodiments, the control circuit is further configured to: transmit an excitation pulse with the radio frequency transmit coil; transmit a first refocusing pulse with the radio frequency transmit coil; apply a first xSPEN readout gradient, during the first refocusing pulse, along a second axis orthogonal to the first axis with the single-sided gradient coil set; apply a second xSPEN readout gradient, during the first refocusing pulse, along a third axis orthogonal to the first axis and orthogonal to the second axis with the single-sided gradient coil set; transmit a second refocusing pulse with the radio frequency transmit coil; apply a third xSPEN readout gradient, during the second refocusing pulse, along the second axis with the single-sided gradient coil set; apply a fourth xSPEN readout gradient, during the second refocusing pulse, along the third axis with the single-sided gradient coil set; and receive data with the radio frequency receive coil. In some embodiments, the control circuit is further configured to: transmit any number of refocusing pulses with the radio frequency transmit coil before, between, or after the refocusing pulses that occur simultaneously with the xSPEN readout gradients. In some embodiments, the control circuit is further configured to: transmit any number of additional xSPEN readout gradients during the first refocusing pulse along any number of additional imaging axes with any number of additional gradient coil sets; transmit any number of additional xSPEN readout gradients during the second refocusing pulse along any number of additional imaging axes with any number of additional gradient coil sets; and transmit a fourth refocusing pulse with the radio frequency transmit coil. In some embodiments, the xSPEN readout axis is not uniform in amplitude across time during the refocusing pulses. In some embodiments, the xSPEN readout axis' gradient shape is not constant in time during the refocusing pulses. In some embodiments, the control circuit is further configured to generate an encoding matrix that is used to generate an image, wherein the encoding matrix accounts for a residual linear phase and a residual quadratic phase. In some embodiments, the control circuit is configured to spatiotemporally encode along an arbitrary axis during an acquisition. In some embodiments, the arbitrary axis is kept fixed during a single image acquisition. In some embodiments, the arbitrary axis is rotated during a single image acquisition.

In another aspect, disclosed herein is a method for generating two dimensional images by collecting a series of projections with varying angles followed by reconstructing the two dimensional images.

In another aspect, disclosed herein is a method for generating three dimensional images by adding an additional gradient pulse array to the pulse sequence. In some embodiments, the additional gradient pulse array is parallel to the xSPEN readout axis and provides spatial information along a permanent gradient axis to be resolved.

In another aspect, disclosed herein is a method for generating three dimensional images with a radial encoding scheme, wherein the effective axis of the gradient array is rotated with the axis of the xSPEN readout gradients.

In another aspect, disclosed herein is a process of applying a plurality of xSPEN readout gradient on the single-sided magnetic resonance imaging system disclosed herein. In some embodiments, applying the plurality of xSPEN readout gradient generates an encoding matrix that is used to generate an image, wherein the encoding matrix accounts for a residual linear phase and a residual quadratic phase. In some embodiments, the process further comprises applying a first xSPEN readout gradient, during a first refocusing pulse, along a second axis orthogonal to a first axis with a single-sided gradient coil set. In some embodiments, the process further comprises applying a second xSPEN readout gradient, during a second refocusing pulse, along the second axis with the single-sided gradient coil set. In some embodiments, the process further comprises applying a third xSPEN readout gradient, during the first refocusing pulse, along the third axis with the single-sided gradient coil set. In some embodiments, the process further comprises applying a fourth xSPEN readout gradient, during the second refocusing pulse, along a third axis with the single-sided gradient coil set.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the various aspects are set forth with particularity in the appended claims. The described aspects, however, both as to organization and methods of operation, may be best understood by reference to the following description, taken in conjunction with the accompanying drawings.

FIG. 1 is a perspective view of a magnetic resonance imaging (MRI) scanner, according to various aspects of the present disclosure.

FIG. 2 is an exploded, perspective view of the MRI scanner of FIG. 1, in which the permanent magnet assembly and the gradient coil sets within the housing are exposed, according to various aspects of the present disclosure.

FIG. 3 is an elevation view of the MRI scanner of FIG. 1, according to various aspects of the present disclosure.

FIG. 4 is an elevation view of the MRI scanner of FIG. 1, according to various aspects of the present disclosure.

FIG. 5 is a perspective view of the permanent magnet assembly of the MRI scanner of FIG. 1, according to various aspects of the present disclosure.

FIG. 6 is an elevation view of the gradient coil set and the permanent magnet assembly of the MRI system shown in FIG. 1, according to various aspects of the present disclosure.

FIG. 7 is a control schematic for a single-sided MRI system, according to various aspects of the present disclosure.

FIG. 8 is a schematic of the magnetic gradient along the Z axis, according to various aspects of the present disclosure.

FIG. 9 is a representative graph of a sweeping frequency pulse, according to various aspects of the present disclosure.

FIG. 10 is a diagram of a pulse sequence for cross-term spatiotemporal encoding for rapid imaging with a single-sided MRI.

FIG. 11 is a diagram of a pulse sequence for cross-term spatiotemporal encoding for radial imaging with a single-sided MRI.

FIG. 12(A) shows the single-sided prostate MRI scanner. The subject sits in lithotomy position and there is a hole in the magnet for surgical and biopsy access to the prostate. FIG. 12(B) shows the xSPEN imaging geometry. Slice-selection is performed along the magnet's z-dimension, and the in-plane (x/y) dimensions are conventionally both phase-encoded.

FIG. 13 illustrates the phase-encoded xSPEN imaging pulse sequence.

FIG. 14 shows the xSPEN spatiotemporal encoding function as it moves along the y-direction during a 1.5 ms-long readout window in a central slice of the in vivo scan. The FOV here was 18×18 cm, and the x-dimension was phase encoded.

FIGS. 15(A) and 15(B) illustrate reconstructions of a center slice of a 10 cm-diameter ACR phantom. FIG. 15(A) was obtained via a Fourier reconstruction described previously. FIG. 15(B) was obtained with the described model-based reconstruction which completely accounts for gradient non-linearity.

FIG. 16(A) illustrates the single-sided MRI scanner. FIG. 16(B) illustrates the radial xSPEN pulse sequence diagram.

FIG. 17(A) shows the numerically calculated radial xSPEN point spread functions, for time points near the start, at the middle, and near the end of the readout. FIG. 17(B) shows middle-readout PSFs at 0, 45, and 90 degree radial angles.

FIG. 18(A) shows the simulated (sinc) radial xSPEN PSF's at three different angles in the middle of their readouts. FIG. 18(B) shows the simulated 45 degree sinc radial xSPEN PSF at three different time points in its readout. The sinc sweeps from the top left of the FOV to the bottom right. FIG. 18(C) shows the phantom images reconstruction with all projections, ¼ projections, and half the readout duration.

FIG. 19(A) shows an xSPEN sinogram of the resolution phantom. The data have the form of an x-ray sinogram, and there is no k-space dimension. FIG. 19(B) are images reconstructed from the sinogram data with three methods, an inverse radon transform that neglects gradient nonlinearity in its initial Fourier transform step, a reconstruction using analytic sinc functions which does not account for Gz nonlinearity, and a reconstruction using the numeric PSF approach illustrated in FIGS. 17(A) and 17(B).

FIG. 20 shows images of the ACR extremity phantom collected with varying fields of view/readout durations.

FIG. 21(A) illustrates the single-sided MRI scanner. FIG. 21(B) illustrates the three dimensional xSPEN pulse sequence, which is based on a CPMG-RARE scan with WURST excitation and refocusing pulses, and which uses the CHORUS technique to place the first spin echo after the second refocusing pulse.

FIG. 22(A) shows the cross-sections of bilinear xSPEN spatial encoding phase functions, through the slab (z) and the xSPEN-encoded in-plane dimension (x), for different x-phase encodes which move the saddle/sensitive point to different z locations through the slab. FIG. 22(B) shows the cross-sections of phase functions for different time points in the readout (with no x-phase encoding), where the saddle/sensitive point shifts to different in-plane (x) locations.

FIG. 23(A) shows a high-resolution xSPEN spatial encoding bilinear phase map (top; cross-section through the slice) and a PSF for each slice (bottom). FIG. 23(B) illustrates that the forward model relating reconstructed images to the data is implemented by applying the point spread functions for each slice and each readout time point to the input image stack, then calculating a Type-III NUFFT to apply phase encoding.

FIG. 24 shows 12 slices from two contiguous slabs of the ACR phantom, reconstructed from 3D xSPEN data. The images show a clear evolution of features between slices, reflecting the sequence's ability to resolve details between within-slab sub-slices.

The accompanying drawings are not intended to be drawn to scale. Corresponding reference characters indicate corresponding parts throughout the several views. For purposes of clarity, not every component may be labeled in every drawing. The exemplifications set out herein illustrate certain embodiments of the disclosure, in one form, and such exemplifications are not to be construed as limiting the scope of the disclosure in any manner.

### DETAILED DESCRIPTION

The following international patent applications are incorporated by reference herein in their respective entireties:
International Application No. PCT/US2020/018352, titled SYSTEMS AND METHODS FOR ULTRALOW FIELD RELAXATION DISPERSION, filed Feb. 14, 2020, published as International Publication No. WO2020/168233;
International Application No. PCT/US2020/019530, titled SYSTEMS AND METHODS FOR PERFORMING MAGNETIC RESONANCE IMAGING, filed Feb. 24, 2020, published as International Publication No. WO2020/172673;
International Application No. PCT/US2020/019524, titled PSEUDO-BIRDCAGE COIL WITH VARIABLE TUNING AND APPLICATIONS THEREOF, filed Feb. 24, 2020, published as International Publication No. WO2020/172672;
International Application No. PCT/US2020/024776, titled SINGLE-SIDED FAST MRI GRADIENT FIELD COILS AND APPLICATIONS THEREOF, filed Mar. 25, 2020, published as International Publication No. WO2020/198395;
International Application No. PCT/US2020/024778, titled SYSTEMS AND METHODS FOR VOLUMETRIC ACQUISITION IN A SINGLE-SIDED MRI SYSTEM, filed Mar. 25, 2020, published as International Publication No. WO2020/198396;
International Application No. PCT/US2020/039667, title SYSTEMS AND METHODS FOR IMAGE RECONSTRUCTIONS IN MAGNETIC RESONANCE IMAGING, filed Jun. 25, 2020, published as International Publication No. WO2020/264194;
International Application No. PCT/US2021/014628, titled MRI-GUIDED ROBOTIC SYSTEMS AND METHODS FOR BIOPSY, filed Jan. 22, 2021; and
International Application No. PCT/US2021/018834, titled RADIO FREQUENCY RECEPTION COIL NETWORKS FOR SINGLE-SIDED MAGNETIC RESONANCE IMAGING, filed Feb. 19, 2021;
International Patent Application No. PCT/US2021/021464, titled PHASE ENCODING WITH FREQUENCY SWEEP PULSES FOR MAGNETIC RESONANCE IMAGING IN INHOMOGENEOUS MAGNETIC FIELDS, filed Mar. 9, 2021;
International Patent Application No. PCT/US2021/021461, titled PULSE SEQUENCES AND FREQUENCY SWEEP PULSES FOR SINGLE-SIDED MAGNETIC RESONANCE IMAGING, filed Mar. 9, 2021;
International Patent Application No. PCT/US2022/071924, titled INTERVENTIONAL LOCALIZATION GUIDE AND METHOD FOR MRI GUIDED PELVIC INTERVENTIONS, filed Apr. 26, 2022; and
International Patent Application No. PCT/US2022/082551, titled RELAXATION-BASED MAGNETIC RESONANCE THERMOMETRY WITH A LOW-FIELD SINGLE-SIDED MRI SCANNER, filed Dec. 29, 2022.

U.S. Patent Application Publication No. 2018/0356480, titled UNILATERAL MAGNETIC RESONANCE IMAGING SYSTEM WITH APERTURE FOR INTERVENTIONS AND METHODOLOGIES FOR OPERATING SAME, published Dec. 13, 2018, is also incorporated by reference herein in its entirety.

It should be noted that the illustrative examples are not limited in application or use to the details of construction and arrangement of parts illustrated in the accompanying drawings and description. The illustrative examples may be implemented or incorporated in other aspects, variations, and modifications, and may be practiced or carried out in various ways. Further, unless otherwise indicated, the terms and expressions employed herein have been chosen for the purpose of describing the illustrative examples for the convenience of the reader and are not for the purpose of limitation thereof. Also, it will be appreciated that one or more of the following-described aspects, expressions of aspects, and/or examples, can be combined with any one or more of the other following-described aspects, expressions of aspects, and/or examples.

In accordance with various aspects, an MRI system is provided that can include a unique imaging region that can be offset from the face of a magnet. Such offset and single-sided MRI systems are less restrictive as compared to traditional MRI scanners. In addition, this form factor can have a built-in or inherent magnetic field gradient that creates a range of magnetic field values over the region of interest. In other words, the inherent magnetic field can be inhomogeneous and can be always active. The inhomogeneity of the magnetic field strength in the region of interest for the single-sided MRI system can be more than 200 parts per million (ppm). For example, the inhomogeneity of the magnetic field strength in the region of interest for the single-sided MRI system can between 200 ppm and 200,000 ppm. In various aspects of the present disclosure, the inhomogeneity in the region of interest can be greater than 1,000 ppm and can be greater than 10,000 ppm. In one instance, the inhomogeneity in the region of interest can be 81,000 ppm.

The inherent magnetic field gradient can be generated by a permanent magnet within the MRI scanner. The magnetic field strength in the region of interest for the single-sided MRI system can be less than 1 Tesla (T), for example. For example, the magnetic field strength in the region of interest for the single-sided MRI system can be less than 0.5 T. In other instances, the magnetic field strength can be greater than 1 T and may be 1.5 T, for example. This system can operate at a lower magnetic field strength as compared to typical MRI systems allowing for a relaxation on the gradient and/or radio frequency coil design constraints and/or allowing for additional mechanisms, like robotics, for example, to be used with the MRI scanner. Exemplary MRI-guided robotic systems are further described in International Application No. PCT/US2021/014628, titled MRI-GUIDED ROBOTIC SYSTEMS AND METHODS FOR BIOPSY, filed Jan. 22, 2021, for example.

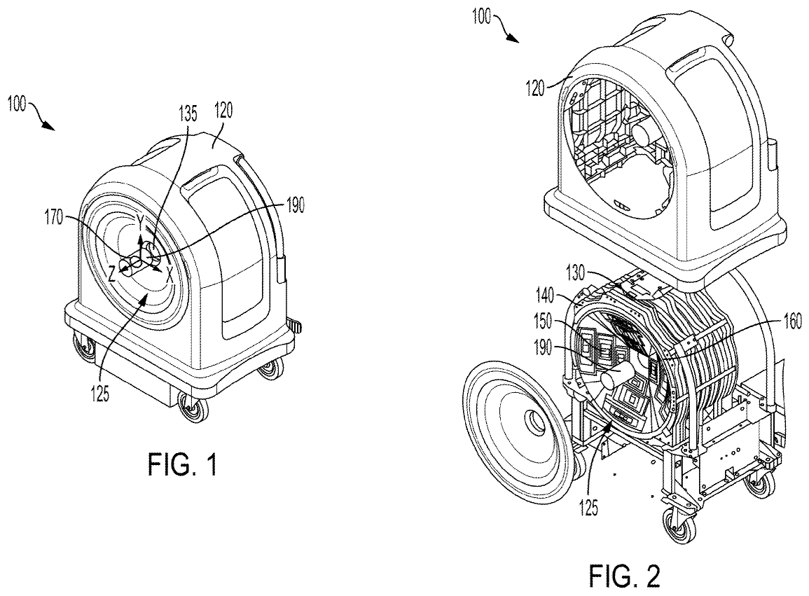

FIGS. 1-6 depict an MRI scanner 100 and components thereof. As shown in FIGS. 1 and 2, the MRI scanner 100 includes a housing 120 having a face or front surface 125, which is concave and recessed. In other aspects, the face of the housing 120 can be flat and planar. The front surface 125 can face the object being imaged by the MRI scanner. As shown in FIGS. 1 and 2, the housing 120 includes a permanent magnet assembly 130, an RF transmission coil (TX) 140, a gradient coil set 150, an electromagnet 160, and a RF reception coil (RX) 170. In other instances, the housing 120 may not include the electromagnet 160. Moreover, in certain instances, the RF reception coil 170 and the RF transmission coil 140 can be incorporated into a combined Tx/Rx coil array. In various instances, the MRI scanner 100 is a single-sided scanner and the various components, e.g., the permanent magnet assembly 130, the RF transmission coil (TX) 140, the gradient coil set 150, the electromagnet 160, and the RF reception coil (RX) 170, are positioned on the same side of the field of view.

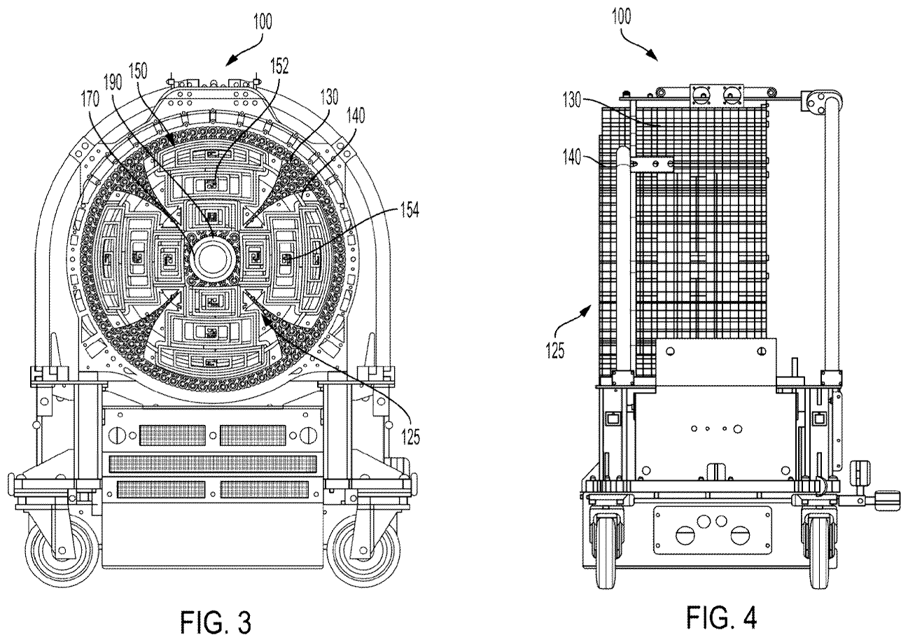

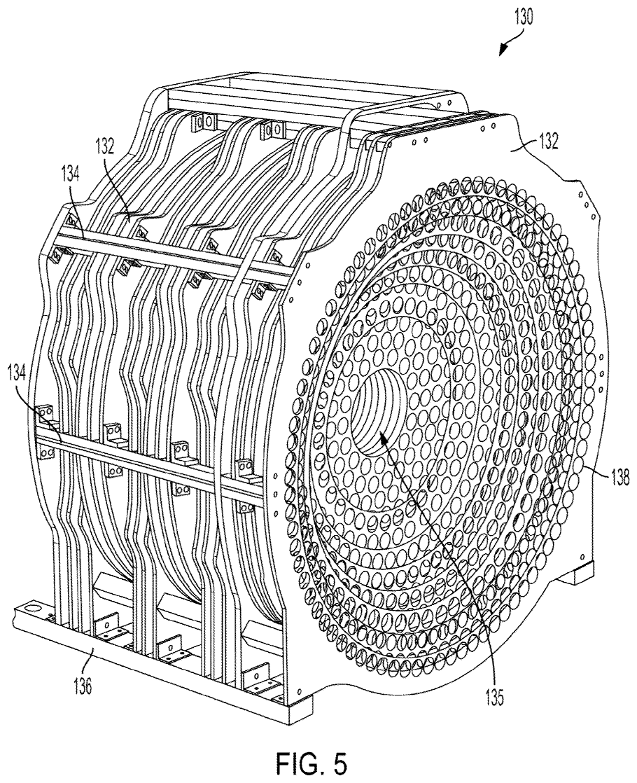

Referring primarily to FIGS. 3-5, the permanent magnet assembly 130 includes an array of magnets. The array of magnets forming the permanent magnet assembly 130 are configured to cover the front surface 125, or patient-facing surface, of the MRI scanner 100 (see FIG. 3) and are shown as horizontal bars in FIG. 4. The permanent magnet assembly 130 includes a plurality of cylindrical permanent magnets in a parallel configuration. Referring primarily to FIG. 5, the permanent magnet assembly 130 comprises parallel plates 132 that are held together by brackets 134. The system can be attached to the housing 120 of the MRI scanner 100 at a bracket 136. There can be a plurality of holes 138 in the parallel plates 132. The permanent magnet assembly 130 can include any suitable magnetic materials, including but not limited to rare-earth based magnetic materials, such as for example, Neodymium-based magnetic materials, for example.

The permanent magnet assembly 130 defines an access aperture or bore 135, which can provide access to the patient through the housing 120 from the opposite side of the housing 120. In other aspects of the present disclosure, the array of permanent magnets forming a permanent magnet assembly in the housing 120 may be bore-less and define an uninterrupted or contiguous arrangement of permanent magnets without a bore defined therethrough. In still other instances, the array of permanent magnets in the housing 120 may form more than one bore/access aperture therethrough.

In accordance with various aspects of the present disclosure, the permanent magnet assembly 130 provides a magnetic field B0 in a region of interest 190 that is along the Z axis, shown in FIG. 1. The Z axis is perpendicular to the permanent magnet assembly 130. Stated differently, the Z axis extends from a center of the permanent magnet assembly 130 and defines a direction of the magnetic field B0 away from the face of the permanent magnet assembly 130. The Z axis can define the primary magnetic field B0 direction. The primary magnetic field B0 can decrease along the Z axis, i.e., an inherent gradient, farther from the face of the permanent magnet assembly 130 and in the direction indicated with the arrow in FIG. 1.

In one aspect, the inhomogeneity of the magnetic field in the region of interest 190 for the permanent magnet assembly 130 can be approximately 81,000 ppm. In another aspect, the inhomogeneity of the magnetic field strength in the region of interest 190 for the permanent magnet assembly 130 can be between 200 ppm to 200,000 ppm and can be greater than 1,000 ppm in certain instances, and greater than 10,000 ppm in various instances.

In one aspect, the magnetic field strength of the permanent magnet assembly 130 can be less than 1 T. In another aspect, the magnetic field strength of the permanent magnet assembly 130 can be less than 0.5 T. In other instances, the magnetic field strength of the permanent magnet assembly 130 can be greater than 1 T and may be 1.5 T, for example. Referring primarily to FIG. 1, the Y axis extends up and down from the Z axis and the X axis extends to the left and right from the Z axis. The X axis, the Y axis, and the Z axis are all orthogonal to one another and the positive direction of each axis is indicated by the corresponding arrow in FIG. 1.

The RF transmission coils 140 are configured to transmit RF waveforms and associated electromagnetic fields. The RF pulses from the RF transmission coils 140 are configured to rotate the magnetization produced by the permanent magnet 130 by generating an effective magnetic field, referred to as B1, that is orthogonal to the direction of the permanent magnetic field (e.g., an orthogonal plane).

Referring primarily to FIG. 3, the gradient coil set 150 includes two sets of gradient coils 152, 154. The sets of gradient coils 152, 154 are positioned on the face or front surface 125 of the permanent magnet assembly 130 intermediate the permanent magnet assembly 130 and the region of interest 190. Each set of gradient coils 152, 154 includes a coil portion on opposing sides of the bore 135. Referring to the axes in FIG. 1, the gradient coil set 154 may be the gradient coil set corresponding to the X axis, for example, and the gradient coil set 152 may be the gradient coil set corresponding to the Y axis, for example. The gradient coils 152, 154 enable encoding along the X axis and Y axis, as further described herein.

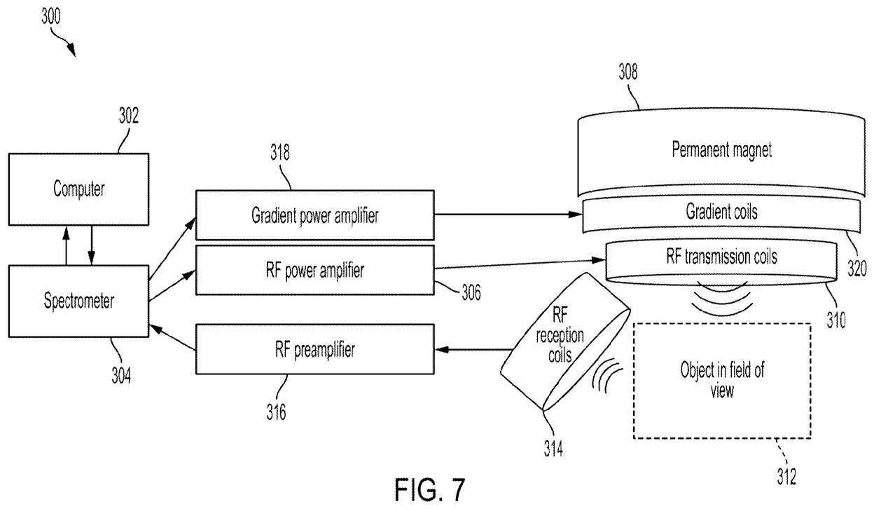

Referring now to FIG. 7, a control schematic for a single-sided MRI system 300 is shown. The single-sided MRI scanner 100 and/or components thereof (FIGS. 1-6) can be incorporated into the MRI system 300 in various aspects of the present disclosure. For example, the imaging system 300 includes a permanent magnet assembly 308, which can be similar to the permanent magnet assembly 130 (see FIGS. 2-5) in various instances. The imaging system 300 also includes RF transmission coils 310, which can be similar to the RF transmission coil 140 (see FIG. 3), for example. Moreover, the imaging system 300 includes RF reception coils 314, which can be similar to the RF reception coils 170 (see FIG. 3), for example. In various aspects, the RF transmission coils 310 and/or the RF reception coils can also be positioned in the housing of an MRI scanner and, in certain instances, the RF transmission coils 310 and the RF reception coils 314 can be combined into integrated Tx/Rx coils. The system 300 also includes gradient coils 320, which are configured to generate gradient fields to facilitate imaging of the object in the field of view 312.

The single-sided MRI system 300 also includes a computer 302, which is in signal communication with a spectrometer 304, and is configured to send and receive signals between the computer 302 and the spectrometer 304.

The main magnetic field B0 generated by the permanent magnet 308 extends away from the permanent magnet 308 and away from the RF transmission coils 310 into the field of view 312. The field of view 312 contains an object that is being imaged by the MRI system 300.

During the imaging process, the main magnetic field B0 extends into the field of view 312. The direction of the effective magnetic field (B1) changes in response to the RF pulses and associated electromagnetic fields from the RF transmission coils 310. For example, the RF transmission coils 310 are configured to selectively transmit RF signals or pulses to an object in the field of view, e.g., tissue. These RF pulses alter the effective magnetic field experienced by the spins in the sample (e.g., patient tissue). RF pulses change the effective magnetic field experienced by the magnetization in the rotating frame. When the RF pulse is on resonance, the effective magnetic field is solely along the axis of the RF pulse. When off resonance, the axis of the effective field will be between the applied RF pulse and the static magnetic field. The RF pulses can be chirp or frequency sweep pulses, for example, as further described herein.

Moreover, when the object in the field of view 312 is excited with RF pulses from the RF transmission coils 310, the precession of the object results in an induced electric current, or MR current, which is detected by the RF reception coils 314. The RF reception coils 314 can send the excitation data to an RF preamplifier 316. The RF preamplifier 316 can boost or amplify the excitation data signals and send them to the spectrometer 304. The spectrometer 304 can send the excitation data to the computer 302 for storage, analysis, and image construction. The computer 302 can combine multiple stored excitation data signals to create an image, for example.

From the spectrometer 304, signals can also be relayed to the RF transmission coils 310 via an RF power amplifier 306, and to the gradient coils 320 via a gradient power amplifier 318. The RF power amplifier 306 amplifies the signal and sends it to RF transmission coils 310. The gradient power amplifier 318 amplifies the gradient coil signal and sends it to the gradient coils 320.

Systems and methods for effectively collecting nuclear magnetic resonance spectra and magnetic resonance images in inhomogeneous fields, such as with the single-sided MRI scanner 100 and system 300, for example, are described herein.

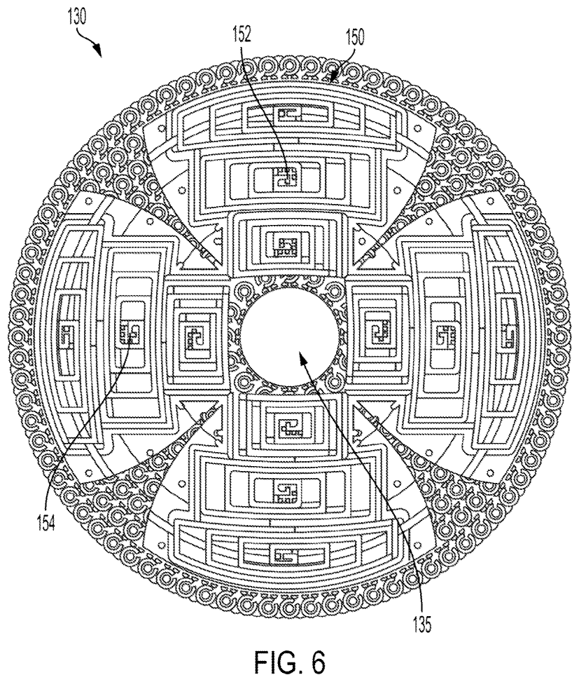

Imaging with a single-sided or open MRI presents many challenges. Typically, two sets of gradient coils (see FIG. 6) in single-sided systems are placed on the face of the permanent magnet assembly. As a result, the amplitude of the gradient will drop as one moves away from the face of the permanent magnet assembly. So, for a given array of phase encodes, the field of view will change as one moves along the axis of the permanent magnetic field B0. In other words, the pulsed gradient coils in a single-sided scanner have a small component along the direction of the permanent gradient.

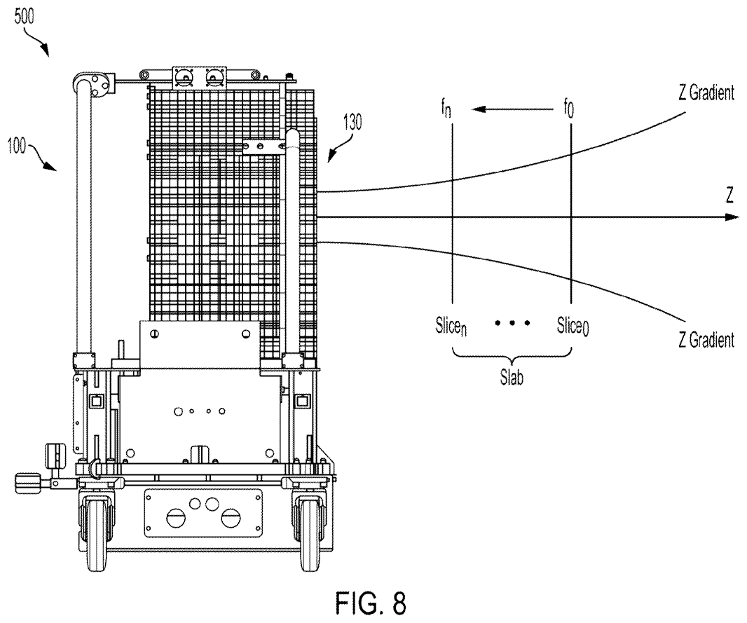

FIG. 8 is a schematic 500 of the magnetic field gradient along the Z axis for the MRI scanner 100. The permanent magnet 130 has an inherent gradient along the Z axis. The strength of the Z gradient decreases as one moves away from the permanent magnet 130. The Z gradient can be seen in the schematic bending away as one moves away from the permanent magnet causing the strength of the gradient to decrease. The MRI scanner 100 images multiple slices to create a slab. Each slice is excited for imaging at a different frequency. The lower frequencies excite tissue for slices farther away from the permanent magnet and higher frequencies excite the tissue in slices closer to the magnet. In the schematic, the slab or axial image is made of multiple slices going from Slice₀ to Sliceₙ. Each slice has a corresponding frequency f₀ to fₙ, where f₀ is a frequency that is smaller than fₙ. It is important to note that the single sided MRI system can be built in such a way that there exists a region where the gradient is inverted from what is described and the frequency can increase along the Z axis.

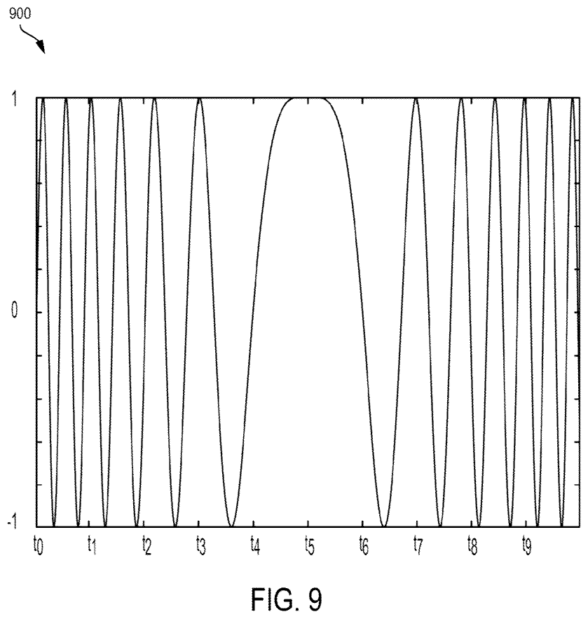

FIG. 9 shows a representative graph 900 of a sweeping frequency pulse or chirp pulse (shown demodulated to a baseband frequency), where the swept direction is set from low to high. A chirped excitation pulse, with the swept direction set from low to high, is an example of a frequency sweep excitation pulse. The frequency of a chirp pulse with the swept direction set from low to high begins at a low frequency and the frequency increases through time for the duration of the pulse. The pulse can begin at the lowest frequency desired and ends once the maximum desired frequency is reached. The pulse frequency in the graph 900 can be a negative-to-positive frequency offset to the baseband frequency. In other words, the frequency sweeps from negative to positive plus the baseband frequency. For example, for a frequency sweep of +/−100 KHz, the sweep is from the baseband frequency less 100 KHz to the baseband frequency plus 100 KHz. It is important to note that the frequency direction might be inverted and sweep from a positive frequency to a negative frequency.

The frequency of a chirp pulse can vary from a minimum (lowest) desired frequency to a maximum (highest) desired frequency. The sweep rate of the pulse is the difference between the highest frequency and lowest frequency in the pulse divided by the time required to go between the highest frequency and the lowest frequency. In one aspect, the frequency range that is covered by the sweeping frequency pulses may be from −20 KHz to 20 KHz, i.e., a 40 KHz range, with a center frequency that varies slab to slab. For example, a slab could be centered at 2.62 MHz, 2.75 MHz, 2.65 MHz, 2.72 MHz, 2.79 MHz, 2.69 MHz, and so on. For a slab centered at 2.62 MHz, the chirp pulse would sweep from 2.60 MHz to 2.64 MHz, i.e., a 40 KHz range. In other aspects of the present disclosure, bandwidths as low as 10 KHz to as high as 200 KHz may be used in the frequency sweep pulse. Moreover, the sweep range can be less than 40 KHz in various instances.

Cross-Term Spatiotemporal Encoding (xSPEN) Techniques for Rapid Imaging with a Single-Sided MRI

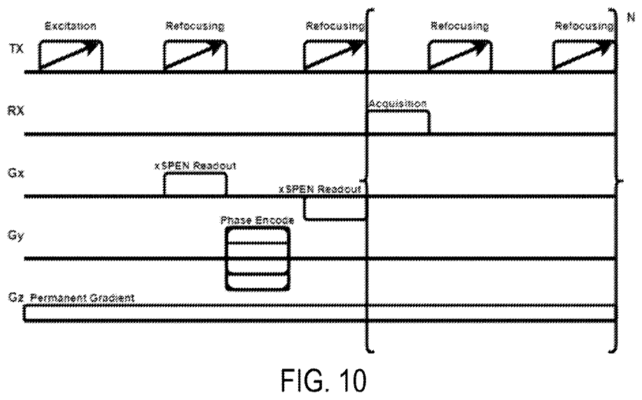

Referring to FIG. 10, the present application discloses a version of xSPEN that can improve SNR and allow for rapid three dimensional image acquisition. In some embodiments, imaging with xSPEN allows for one of the axes in an image to be rapidly sampled, cutting down on scan time significantly. Adapting xSPEN to a single sided low field system requires some changes to the design of the pulse sequence.

First, the rapid sequence can include a chirped excitation pulse to collect a thicker slab. By collecting a thicker slab, the SNR can be increased because more volume, and therefore more magnetization, contributes to the signal. The conventional xSPEN sequence uses a hard pulse to excite magnetization because it was designed for systems with much weaker gradients. Under those conditions, a hard pulse is capable of exciting a slice thick enough to generate sufficient SNR. In some embodiments, the pulse sequence may be modified to remove the quadratic phase imposed on the magnetization by the chirped excitation by using the timing. See Foroozandeh, Mohammadali, et al. “Improved ultra-broadband chirp excitation.” Journal of Magnetic Resonance, vol. 302, 2019, pp. 28-33. and Power, J. E., et al, “Increasing the quantitative bandwidth of NMR measurements.” The Royal Society of Chemistry, Chem. Commun., vol. 52, 2016, pp. 2916-2919, each of which is incorporated by reference herein in its entirety.

In some embodiments, to further increase the SNR, multiple echoes can be collected with the xSPEN sequence. Like the other pulse sequences that use chirped refocusing pulses, every other echo can be discarded because they retain the quadratic phase imparted onto it by the chirped pulses. By collecting multiple echoes per acquisition, the present applicant demonstrates how to signal average without having to wait for the magnetization to return to the longitudinal axis.

In another implementation, each echo can be used to sample a different line in k space. Before collecting an echo, a gradient can be used to move the magnetization to a different point in k space, allowing for multiple k-spaces lines to be collected during an excitation, rapidly speeding up image acquisition.

Some embodiments of the rapid xSPEN sequences described herein may contain interleaved slices to collect three dimensional information. These scans can be designed to monitor changes that occur in all three dimensions, such as the movement of a biopsy needle in a patient or the formation of an ice ball during a cryo ablation experiment, so they have to generate volumetric images. In some embodiment the volumes are generated by collecting a series of two dimensional images in the x/y plane along the z axis. The images can then be interpolated along the z axis to make a full volume.

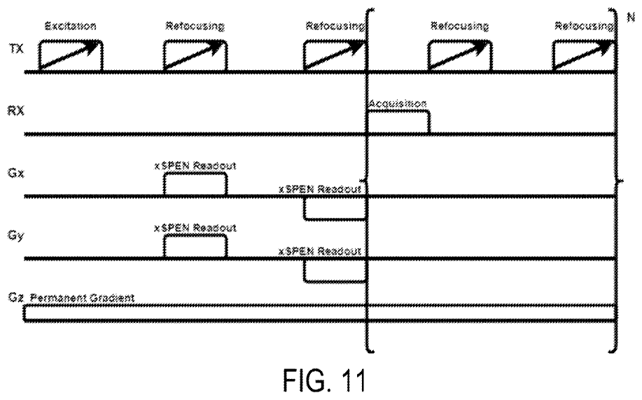

Another embodiment of the rapid xSPEN sequences described herein may contain a phase encoded third axis. This allows for thick excitation profiles to be acquired to leverage the benefits of increased SNR while simultaneously encoding image information across that axis. This image is shown in FIG. 11. This allows one to collect volumetric images with an xSPEN sequence. One version of this technique uses two gradient pulse arrays to encode spatial information along two axes, while the third is read out during the acquisition period. One gradient pulse array is along an axis perpendicular to readout axis while the other is along the same axis. The gradient pulse array perpendicular to the readout axis is used to phase encode spatial information. The other array spatiotemporally encodes information, but not along the axis of the gradients, but instead along the axis of the other gradient that is on during the two chirped pulses used to impart a hyperbolic phase onto the magnetization. So, with one phase encode array, one other array that encodes spatial information along one axis, and a readout along another axis, all three axes can be encoded, allowing for one to collect volumetric images with xSPEN.

In another aspect, another way that three dimensional volumes can be collected with xSPEN is with a radial encoding scheme that is paired with a gradient array. Encoding along two axes can be achieved by collecting projections along various angles, until projections along enough angles are collected to reconstruct a two dimensional image. The third dimension is encoded by adding a gradient pulse array to a two dimensional radial xSPEN procedure. The direction of the gradient pulse array is rotated with the angle of the projection, so that a plane along the third axis and the angle of the projection axis is collected. By collecting multiple angles with this method, a three dimensional image can be reconstructed. This method produces an MRI image with no Fourier encoding, though Fourier phase encoding can be additionally applied along the rotating xSPEN-encoded axis to increase spatial resolution without the conventional xSPEN resolution-SNR tradeoff.

### Range of Parameters

Potential range of field inhomogeneities are:
10 to 300 kHz over a 160 mm depth.

Potential range of excitation bandwidths are:
5 to 200 kHz

Potential range of refocusing bandwidths are:
5 to 200 kHz.

Potential range of times to acquire an image are:
1 second to 30 minutes.

In another aspect, the present application provides Cartesian xSPEN sequences. In a Cartesian xSPEN sequence, one axis is spatiotemporally encoded while the other is Fourier encoded. Due to how spatial information is encoded with xSPEN, it is possible to encode a field of view along the spatiotemporal axis that is smaller than the size of the object being imaged. In conventional MRI scans, where both axes are Fourier encoded, choosing a field of view that is smaller than the object results in aliasing. Because xSPEN uses a different encoding method, it is possible to encode a smaller field of view, allowing a user to zoom in one axis.

In some embodiments, the present application discloses a method that allows one to zoom in along both axes thereby cropping the field of view by using only a readout gradient and no other tools, such as a saturation band. Typically, only the spatiotemporal axis may have a field of view smaller than the object size. In applications where only a part of the field of view has useful anatomical information, it would be ideal if the field of view could be cropped in both axes so that the image is zoomed in to the relevant anatomy.

Referring to FIG. 11, a radial xSPEN pulse sequence generates images by collecting a series of projections with rotating axes. These different projections are then combined into a single two dimensional image using an iterative conjugate gradient reconstruction or a filtered back reconstruction approach. Unlike the xSPEN sequences described in the literature, a radial xSPEN sequence has gradients applied during the first two chirped refocusing pulses for both axes, leaving neither of these two axes Fourier encoded. The axis of projection can be altered by adjusting the relative strength of the gradient pulses applied in each axis. When a gradient is applied solely to one axis, the projection will be solely along that axis. If some power is applied to both axes, then the axis of the projection will be between the two axes. By imaging this way, it is possible to collect a two dimensional image that is entirely spatiotemporally encoded. By excluding Fourier encoding from the 2D image acquisition, it is possible to collect an image with a field of view that is smaller than the object without aliasing. In some embodiments, this reduction in the field of view is acquired by truncating the readout axis, which may lead to shorter echo times and higher signal strength due to the reduced transverse relaxation.

In another embodiment, the geometric center of this reduced field of view can be moved by adjusting the schedule of readout angles made between the two axis to a non-uniform sampling. It is also possible to adjust both the schedule of angles and the readout truncation to create an elliptical field of view.

In another embodiment, it is possible to add a small amount of Fourier encoding perpendicular to the xSPEN band's travel direction, to resolve the reduced field of view more accurately across the xSPEN projection. An example of this encoding scheme would be to apply a rotating Fourier phase encoding along each rotating xSPEN projection to increase spatial resolution.

The third dimension for volumetric encoding can be achieved in a variety of ways. The first would be to collect an array of 2D images sliced along a third dimension that comprise a volume. These slices would be acquired by changing the excitation frequency and band or by adding a slice selective electromagnetic gradient pulse during the excitation which shifts the Larmor frequency of the desired volume subset into the desired range. In some embodiments, the second method may add a third xSPEN readout axis using an additional electromagnetic gradient. In this way, the image projection can be tilted in the third dimension allowing for the third imaging axis to be encoded. In some embodiments, the third method may excite the entire desired volume and implement a phase encoding axis on the third dimension.

As the instantaneous amplitude of the xSPEN readout axis adjusts the speed at which the encoding point spread function moves across the image. By adjusting the amplitude and therefore the speed, it could be used to preferentially increase the SNR of segments along the readout projection. Additionally, if the xSPEN readout axis gradient shape is dynamically changed (e.g., by the addition of another controllable readout axis), then the point spread function size can be dynamically adjusted and the pixel resolution across the image can be changed.

### Range of Parameters

Potential range of field inhomogeneities are:
10 to 300 kHz over a 160 mm depth.

Potential range of excitation bandwidths are:
5 to 200 kHz

Potential range of refocusing bandwidths are:
5 to 200 kHz.

Potential range of times to acquire an image are:
1 second to 30 minutes.
   Conjugate Gradient Least Squares Reconstruction for Cross-Term Spatiotemporal Encoding (xSPEN) with a Single-Sided MRI

For Cartesian xSPEN reconstruction, the gradient fields used for image encoding are linear. When the gradient fields are linear, converting xSPEN data to an image can be as simple as taking a Fourier transform along the Fourier encoded direction and taking the absolute value of the resulting image.

As for radial xSPEN, it is not known how to reconstruct data collected using a radial xSPEN sequence. the data should be able to be converted to an image using techniques similar to those used for reconstructing x-Ray images; however, this has not been reported yet.

In this regard, none of the existing reconstruction methods are capable of handling nonlinear gradient fields. If the fields are nonlinear, then the images generated with the existing reconstruction may be distorted. No distortions must be present in the image if they are to be used in clinical diagnoses yet.

In one aspect, the present application describes a conjugate gradient least squares reconstruction that uses maps of the gradient fields to correct the distortions produced by the nonlinearity of gradient coils. This method is based on the signal equation for magnetization generated with an xSPEN sequence, e.g., as described in regard to FIG. 10. With this equation, the different contributions to the phase can be calculated and used to solve for an image without distortions. This method may also be used for reconstructing radial xSPEN images, e.g., as described in regard to FIG. 11.

In some aspects, there are two parts to the reconstruction that need to be considered: the ideal nonlinear xSPEN reconstruction and the residual phase terms. The ideal nonlinear reconstruction can be based on equation one. The time domain signal collected is a one dimensional profile along the spatiotemporal direction, spatially encoded with the xSPEN readout gradients applied during the first two refocusing pulses. In this example, the spatiotemporal axis is along y, but it may be along any direction. In the below equation, the other axis is assumed to be Fourier encoded. An encoding matrix is calculated using these equations. The matrix can then be inverted to recover an undistorted image.

$$S ( t ) α {\int }_{x y} \text{?} dx dy p ( x , y ) {e}^{- i 2 π Gx ( x , y ) PE} \frac{2 π BWex}{{Y}^{G} z ( x , y )} Sinc \{ \frac{π}{BWex} ( - \frac{2 YbGy ( x , y )}{π BWex} + t - \frac{Ta}{2} ) \} \qquad ( 1 )$$

$$\text{?} \text{indicates text missing or illegible when filed}$$

S(t): Time domain signal that is acquired with the scanner
p(x, y): spatial distribution of signal. This is the image that is recovered with the reconstruction
Gₓ(x, y): Magnetic field produced by the x gradient coil
PE: Integral of the phase encoding gradient for this particular line
BWₑₓ: Bandwidth of the excitation pulse used in the pulse sequence
G_{z}(x, y): Magnetic field produced by the array of permanent magnets
B: Integral of the xSPEN readout gradient
Y: Gyromagnetic ratio
G_{y}(x, y): Magnetic field produced by the y gradient coil
T: time in the acquisition window
Tₐ: Duration of the acquisition window

Equation (1) is exact if the first two chirped refocusing pulses are equal in length and if the gradients applied during those pulses are identical. However, if the pulse used for excitation is also a chirped pulse, then the two subsequent pulses will not be the same duration. Their difference in duration can depend on the parameters of the pulse sequence. The existence of a difference results in residual quadratic and linear phase, which can affect image quality, especially in radial reconstructions, where the method may outright fail to produce an image if the residual phase terms are not accounted for.

The two residual phase terms are the quadratic term, shown in Equation (2) and the linear term shown in Equation (3). The residual phase terms are multiplied into Equation (1), which is then used to generate an encoding matrix that can be inverted to solve for an image.

$${ϕ}_{quadratic} = ( 1 - CF ) \frac{{T}^{π}}{2 π BW} {( {YG}_{z} )}^{2} + {P}_{2}^{2} - {CFP}_{1}^{2} ) \frac{{T}^{π}}{2 π BW} {( {YG}_{y} )}^{2} - 2 ( {P}_{2} + {CFP}_{1} ) \frac{{T}^{π}}{2 π BW} {Y}^{2} {G}_{y} {G}_{z} \qquad ( 2 )$$

CF: CHORUS factor which modifies the duration of the first refocusing pulse and the excitation pulse
T^{π}: Duration of the refocusing pulse
BW: Bandwidth of the refocusing pulse
G_{z} Magnetic field produced by the array of permanent magnets
P₁: Strength of the xSPEN readout pulse applied during the first refocusing pulse
P₂: Strength of the xSPEN readout pulse applied during the second refocusing pulse
Y: gyromagnetic ratio
G_{y}: Magnetic field produced by the y gradient coil

$${ϕ}_{linear} = 2 {T}^{π} ( CF - 1 ) {YG}_{z} + 2 {T}^{π} ( {P}_{1} CF + {P}_{2} ) {YG}_{y} \qquad ( 3 )$$

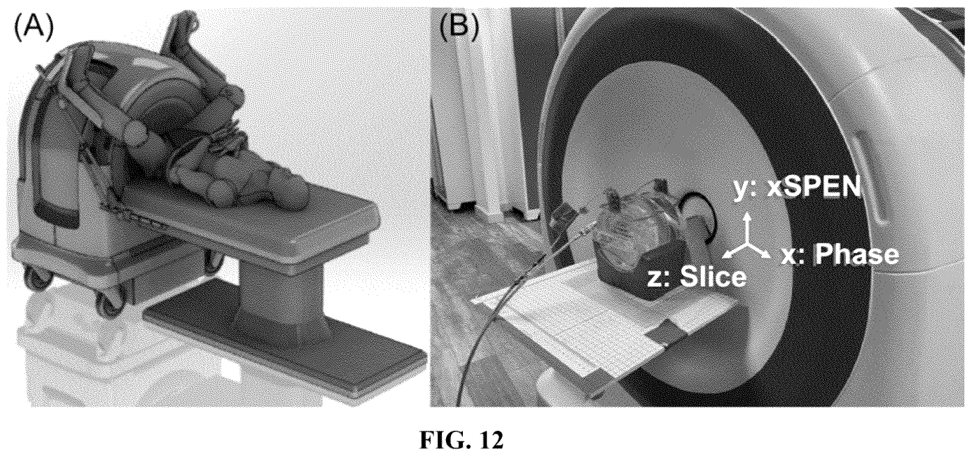

Referring primarily to FIG. 12(B), xSPEN was applied to swap the typically frequency encoded but small matrix dimension (z) with a typically phase-encoded but large matrix dimension (y), for faster imaging.

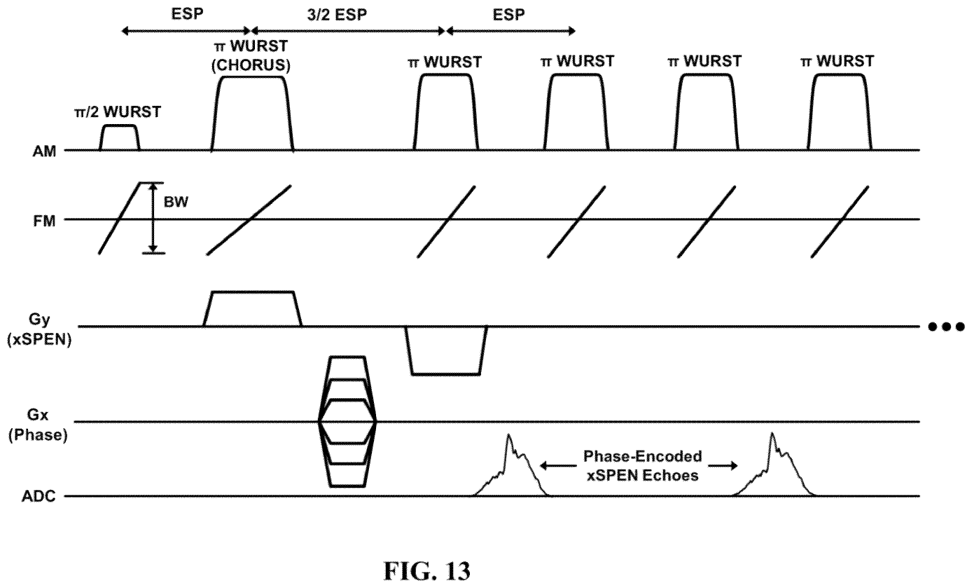

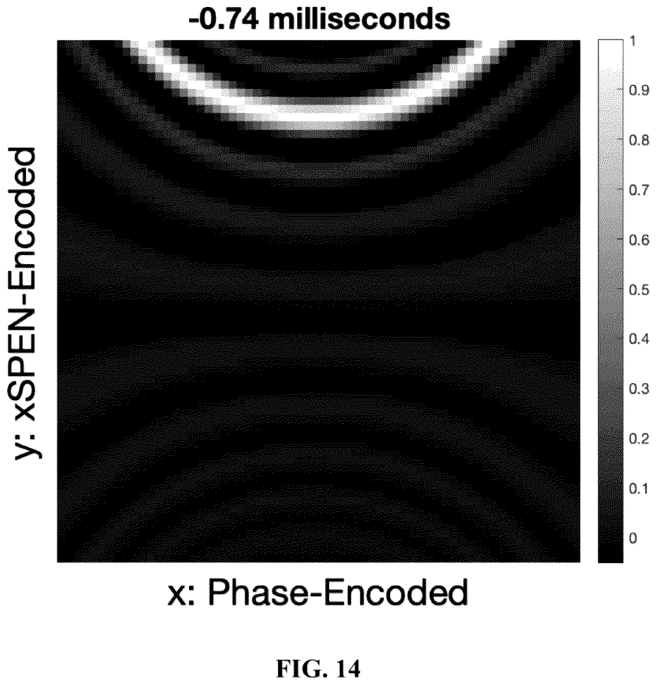

Referring primarily to FIG. 13, chirped WURST pulses were used throughout to excite and refocus the slice's wide bandwidth. The first refocusing pulse is 1.1× longer than subsequent refocusing pulses to compensate both the subsequent pulse's quadratic phase, and the excitation pulse's quadratic phase, yielding a spin echo signal after the second refocusing pulse. This necessitates increasing the amplitude of the second Gy pulse to maintain the same amplitude-pulse duration product which sets the xSPEN bilinear curvature.

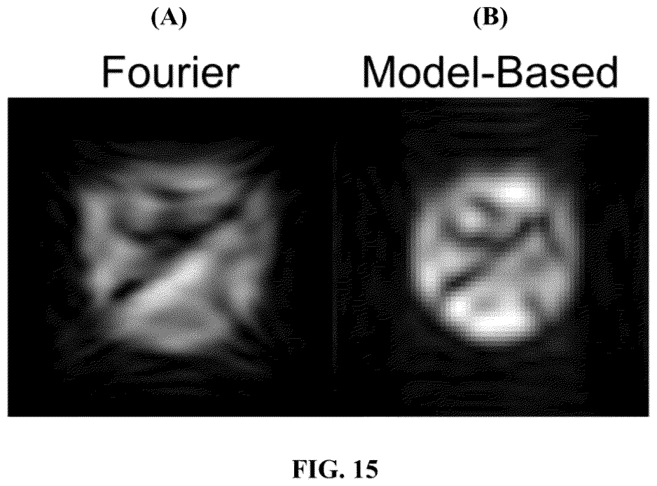

Referring primarily to FIG. 15(B), the model-based reconstruction has significantly lower geometric distortions and more uniform signal inside the phantom.

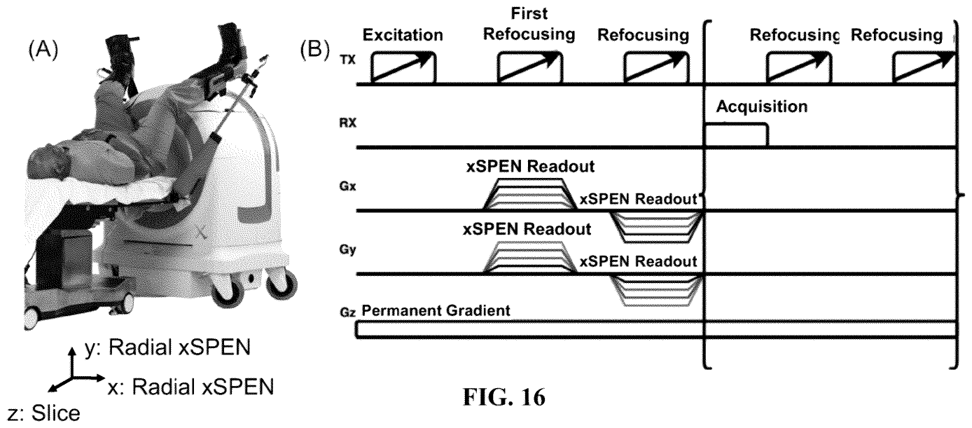

Referring primarily to FIG. 16(B), the sequence is a CPMG acquisition with the CHORUS method to generate a spin echo after the second WURST refocusing pulse, and the section in brackets is repeated 12 times. The xSPEN encoding gradients are applied with opposite polarities during the first two refocusing pulses; different colors represent different in-plane radial angles.

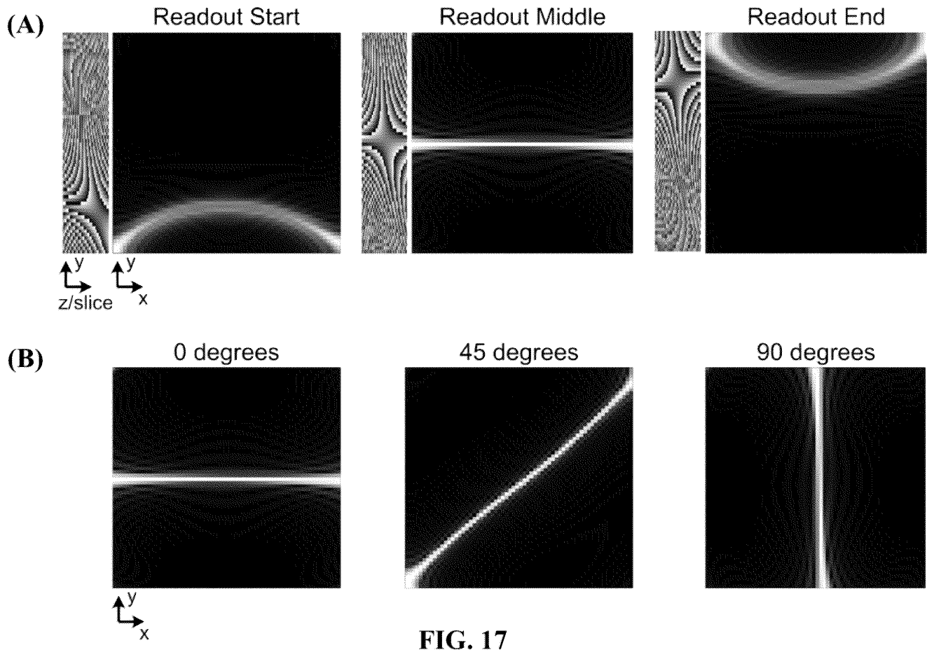

Referring primarily to FIG. 17(A), the numerical PSFs are constructed by summing the Gz-shifted bilinear phase profiles through the slice dimension; central sections of these phase profiles are shown to the left of each calculated in-plane PSF, to illustrate how the bilinear saddle point shifts during the readout, yielding a corresponding shift in the main lobe of the PSF.

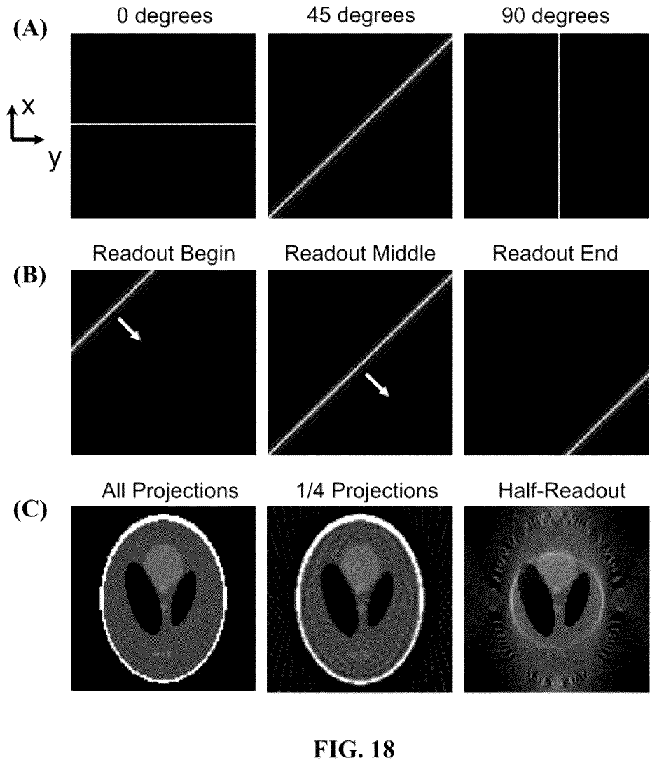

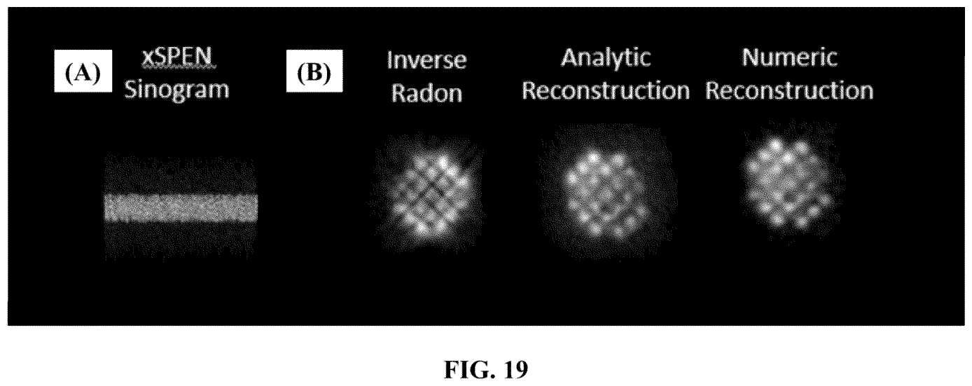

Referring primarily to FIG. 18(C), undersampling the number of projections leads to the same streaking artifacts seen in conventional Fourier radial sampling, but truncating the readout leaves a fully resolved image in the center of the FOV, rather than reduced spatial resolution.

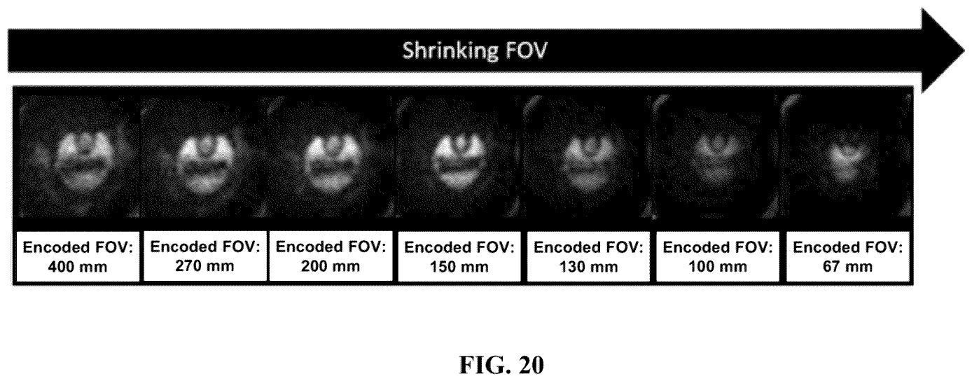

Referring primarily to FIG. 20, the phantom was surrounded by material meant to mimic tissue. As the field of view was reduced, the signal present in the image decreased too, cropping the image. At the end, the field of view was smaller than the phantom. This also enabled the use of shorter echo spacings, in this case a reduction from 5.4 to 3.0 ms which reduced the overall 24-echo readout train duration from 130 ms to 72 ms.

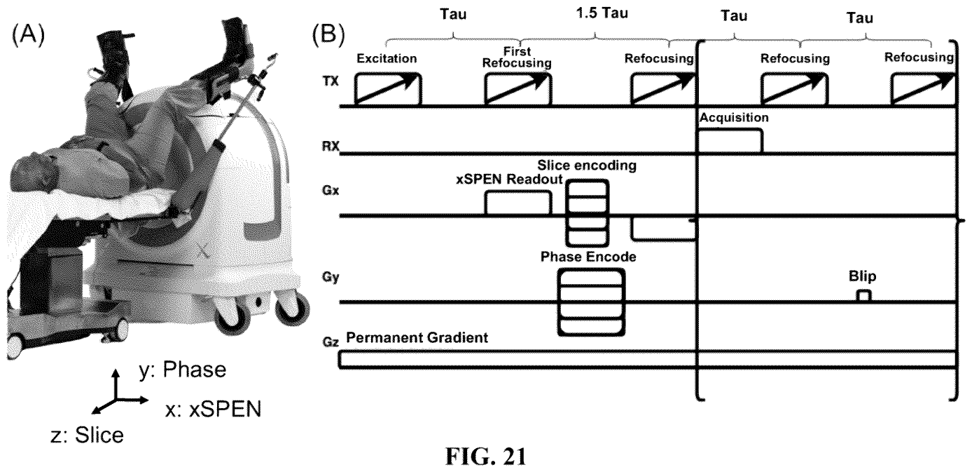

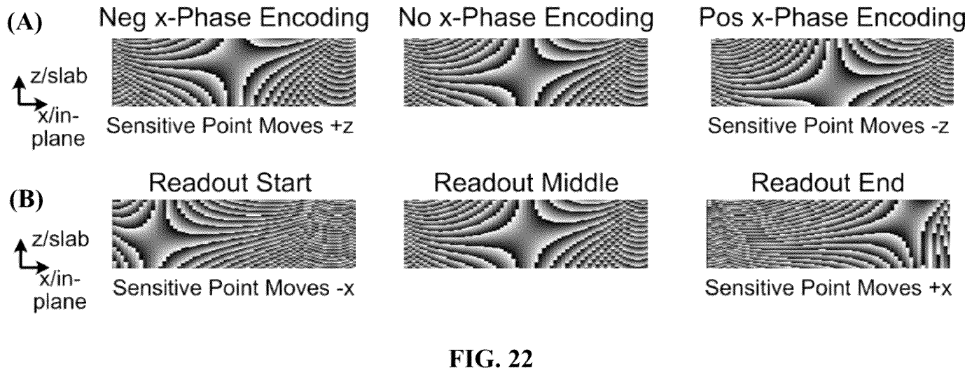

Referring primarily to the diagram of FIG. 21(B), the y axis is Fourier encoded while the x and z axes are spatiotemporally encoded. A profile along x is collected during the readout while the z profile is collected indirectly.

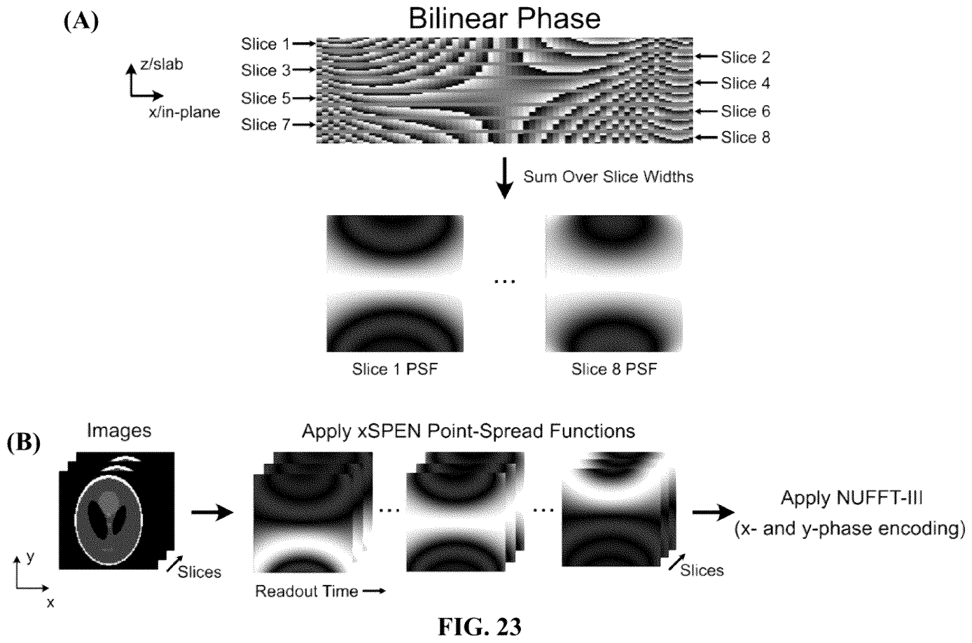

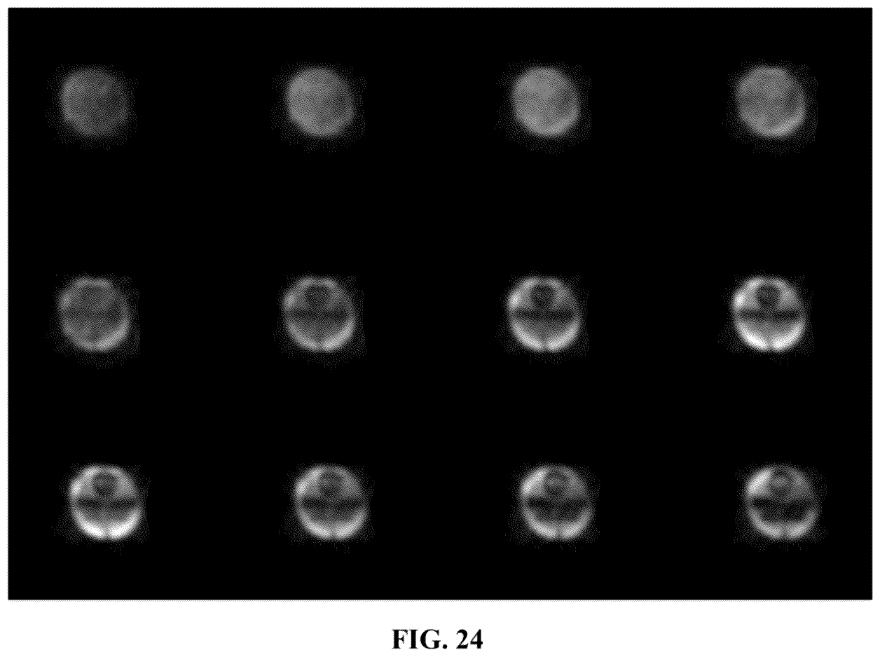

Referring primarily to FIG. 23(A), to calculate the point-spread function (PSF) for each sub-slice in the volume, a high-resolution xSPEN spatial encoding bilinear phase map is calculated and a sum is taken over each sub-slice width, yielding a PSF for each slice.

### Configurations

In some aspects, this reconstruction may be used for any xSPEN sequence, whether it is Cartesian or Radial. It can be used with xSPEN sequences with identical refocusing pulses or with differing refocusing pulses, with the residual phase produced by the differing pulses accounted for with Equations (2) and (3). This reconstruction can also accommodate a RARE style image acquisition. The reconstruction may be run in the following configurations: with just the ideal signal from Equation (1), with the ideal signal and the residual linear phase, with the ideal signal and the quadratic phase, and lastly with the ideal signal and both the residual quadratic and linear phase. In the equations shown above, the spatiotemporal axis was y, and the Fourier axis was x but they can be switched. Alternatively, the Equation (1) can be calculated to have the x and y axes be spatiotemporal axes and no Fourier axis, as described above, in regard to FIG. 11. Or all three axes can use Equation (1) to achieve a three dimensional radial xSPEN sequence reconstruction.

In some aspects, many embodiments of this reconstruction are possible which balance accuracy with computational speed and memory requirements. For 2D reconstruction, a more accurate reconstruction that does not presume linearity of the permanent gradient is obtained by directly constructing a high-resolution three-dimensional phase matrix for each time point in the readout, and then summing that matrix in the slice dimension to obtain a numerical point spread function for each time point in the readout. That point spread function can then be reformed into a matrix and the image can be solved by regularized matrix pseudoinverse, conjugate gradients, or using any other iterative optimization algorithm.

In some aspects, this same approach also applies to the radial reconstruction scenario, in which case the PSF rotates between repetitions. Any applied Fourier phase encoding in any dimension can be implemented as a separate explicit matrix multiplication or can be implemented via a non-uniform Fast Fourier Transform (NUFFT) before or after applying the point spread function matrix. Instead of summing the bilinear phase through the slice, the NUFFT could also be used to apply the permanent gradient-induced phase shift at each time point in the readout, followed by a summing operator applied across the slice. This would greatly reduce the memory size of the PSF matrix.

In some aspects, 3D reconstruction is implemented using a similar approach in which the bilinear phase is directly calculated with high spatial resolution and then summed to obtain a PSF for each sub-slice of the 3D volume and for each time point in the readout. An NUFFT then can be applied for Fourier phase encoding as in the 2D case. Also, as in the 2D case, the memory requirements can be greatly reduced by using the NUFFT to apply the readout phase shifts, followed by a sub-slice summing operator.

### Process

In another aspect, provided herein is a process of applying a plurality of xSPEN readout gradient on the single-sided magnetic resonance imaging system disclosed herein.

In some embodiments, applying the plurality of xSPEN readout gradient generates an encoding matrix that is used to generate an image, wherein the encoding matrix accounts for a residual linear phase and a residual quadratic phase. In some embodiments, applying the plurality of xSPEN readout gradient generates an encoding matrix that is used to generate an image. In some embodiments, the encoding matrix accounts for a residual linear phase and a residual quadratic phase. In some embodiments, the encoding matrix accounts for a residual linear phase. In some embodiments, the encoding matrix accounts for a residual quadratic phase.

In some embodiments, the plurality of xSPEN readout gradient disclosed herein comprises a first xSPEN readout gradient, a second xSPEN readout gradient, a third xSPEN readout gradient, and a fourth xSPEN readout gradient. In some embodiments, the plurality of xSPEN readout gradient disclosed herein comprises a first xSPEN readout gradient, a second xSPEN readout gradient, and a third xSPEN readout gradient. In some embodiments, the plurality of xSPEN readout gradient disclosed herein comprises a first xSPEN readout gradient and a second xSPEN readout gradient. In some embodiments, the plurality of xSPEN readout gradient disclosed herein comprises a first xSPEN readout gradient.

In some embodiments, the process disclosed herein further comprises applying a first xSPEN readout gradient, during a first refocusing pulse, along a second axis orthogonal to a first axis with a single-sided gradient coil set.

In some embodiments, the process disclosed herein further comprises applying a second xSPEN readout gradient, during a second refocusing pulse, along the second axis with the single-sided gradient coil set.

In some embodiments, the process disclosed herein further comprises applying a third xSPEN readout gradient, during the first refocusing pulse, along the third axis with the single-sided gradient coil set.

In some embodiments, the process disclosed herein further comprises applying a fourth xSPEN readout gradient, during the second refocusing pulse, along a third axis with the single-sided gradient coil set.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

1. A single-sided magnetic resonance imaging system comprising:
a housing comprising:
a front surface;
a permanent magnet for providing a static magnetic field that extends from the permanent magnet relative to a first axis into a region of interest, wherein the first axis is perpendicular to the permanent magnet;
a radio frequency transmit coil; and
a single-sided gradient coil set, wherein the radio frequency transmit coil and the single-sided gradient coil set are positioned proximate to the front surface;
a radio frequency receive coil;
a power source, wherein the power source is configured to flow current through at least one of the radio frequency transmit coil or the single-sided gradient coil set to generate an electromagnetic field in the region of interest, wherein at least a portion of the region of interest resides outside the front surface; and
a control circuit configured to:
transmit an excitation pulse with the radio frequency transmit coil;
transmit a first refocusing pulse with the radio frequency transmit coil;
apply a first xSPEN readout gradient, during the first refocusing pulse, along a second axis orthogonal to the first axis with the single-sided gradient coil set;
perform phase encoding along a third axis orthogonal to the first axis and orthogonal to the second axis with the single-sided gradient coil set;
transmit a second refocusing pulse with the radio frequency transmit coil;
apply a second xSPEN readout gradient, during the second refocusing pulse, along the second axis with the single-sided gradient coil set; and
receive data with the radio frequency receive coil.

2. The single-sided magnetic resonance imaging system of claim 1, wherein the control circuit is further configured to:
transmit any number of refocusing pulses with the radio frequency transmit coil before, between, or after the refocusing pulses that occur simultaneously with the xSPEN readout gradients.

3. The single-sided magnetic resonance imaging system of claim 1, wherein the control circuit is further configured to:
generate an encoding matrix that is used to generate an image, wherein the encoding matrix accounts for a residual linear phase and a residual quadratic phase.

4. The single-sided magnetic resonance imaging system of claim 1, wherein the control circuit is further configured to:
transmit an excitation pulse with the radio frequency transmit coil;
transmit a first refocusing pulse with the radio frequency transmit coil;
apply a first xSPEN readout gradient, during the first refocusing pulse, along a second axis orthogonal to the first axis with the single-sided gradient coil set;
apply a second xSPEN readout gradient, during the first refocusing pulse, along a third axis orthogonal to the first axis and orthogonal to the second axis with the single-sided gradient coil set;
transmit a second refocusing pulse with the radio frequency transmit coil;
apply a third xSPEN readout gradient, during the second refocusing pulse, along the second axis with the single-sided gradient coil set;
apply a fourth xSPEN readout gradient, during the second refocusing pulse, along the third axis with the single-sided gradient coil set; and
receive data with the radio frequency receive coil.

5. The single-sided magnetic resonance imaging system of claim 4, wherein the control circuit is further configured to:
transmit any number of refocusing pulses with the radio frequency transmit coil before, between, or after the refocusing pulses that occur simultaneously with the xSPEN readout gradients.

6. The single-sided magnetic resonance imaging system of claim 4, wherein the control circuit is further configured to:
transmit any number of additional xSPEN readout gradients during the first refocusing pulse along any number of additional imaging axes with any number of additional gradient coil sets;
transmit any number of additional xSPEN readout gradients during the second refocusing pulse along any number of additional imaging axes with any number of additional gradient coil sets; and
transmit a fourth refocusing pulse with the radio frequency transmit coil.

7. The single-sided magnetic resonance imaging system of claim 1, wherein the xSPEN readout axis is not uniform in amplitude across time during the refocusing pulses.

8. The single-sided magnetic resonance imaging system of claim 1, wherein the xSPEN readout axis' gradient shape is not constant in time during the refocusing pulses.

9. The single-sided magnetic resonance imaging system of claim 4, wherein the control circuit is further configured to generate an encoding matrix that is used to generate an image, wherein the encoding matrix accounts for a residual linear phase and a residual quadratic phase.

10. The single-sided magnetic resonance imaging system of claim 4, wherein the control circuit is configured to spatiotemporally encode along an arbitrary axis during an acquisition.

11. The single-sided magnetic resonance imaging system of claim 10, wherein the arbitrary axis is kept fixed during a single image acquisition.

12. The single-sided magnetic resonance imaging system of claim 10, wherein the arbitrary axis is rotated during a single image acquisition.

13.-16. (canceled)

17. A process of applying a plurality of xSPEN readout gradient on the single-sided magnetic resonance imaging system of claim 1.

18. The process of claim 17, wherein applying the plurality of xSPEN readout gradient generates an encoding matrix that is used to generate an image, wherein the encoding matrix accounts for a residual linear phase and a residual quadratic phase.

19. The process of claim 17, further comprising applying a first xSPEN readout gradient, during a first refocusing pulse, along a second axis orthogonal to a first axis with a single-sided gradient coil set.

20. The process of claim 19, further comprising applying a second xSPEN readout gradient, during a second refocusing pulse, along the second axis with the single-sided gradient coil set.

21. The process of claim 20, further comprising applying a third xSPEN readout gradient, during the first refocusing pulse, along the third axis with the single-sided gradient coil set.

22. The process of claim 21, further comprising applying a fourth xSPEN readout gradient, during the second refocusing pulse, along a third axis with the single-sided gradient coil set.
